(19) United States
(12) Patent Application Publication
(10) Pub. No.: US 2013/0090550 A1
(43) Pub. Date: Apr. 11, 2013

(54) METHODS OF IDENTIFYING PATIENTS WITH OVARIAN EPITHELIAL NEOPLASMS BASED ON HIGH-RESOLUTION MASS SPECTROMETRY

(71) Applicants: Elvio G. Silva, Houston, TX (US); Cristina Fente Sampayo, Lugo (ES)

(72) Inventors: Elvio G. Silva, Houston, TX (US); Cristina Fente Sampayo, Lugo (ES)

(73) Assignee: BOARD OF REGENTS, THE UNIVERSITY OF TEXAS SYSTEM, Austin, TX (US)

(21) Appl. No.: 13/703,444

(22) PCT Filed: Jun. 9, 2011
(86) PCT No.: PCT/US11/39831

§ 371 Date: Dec. 11, 2012

Related U.S. Application Data
(60) Provisional application No. 61/353,753, filed on Jun. 11, 2010.

(51) Int. Cl. G01N 33/68; A61B 10/02; A61B 5/055; A61B 8/12; A61B 6/03

(52) U.S. Cl. CPC G01N 33/689; A61B 8/12; A61B 6/03; A61B 5/055; A61B 10/02
USPC 600/411; 436/86; 436/87; 530/324; 600/437; 600/427; 600/562

(57) ABSTRACT

Methods for identifying patients with ovarian neoplasms are provided herein comprising performing an assay step of detecting a metabolite in a serum sample of a patient where the metabolite is probably not produced by a tumor but could induce the development of a tumor, and the presence of the metabolite being indicative of an increased likelihood that the patient has ovarian cancer,

### CROSS REFERENCE TO RELATED APPLICATIONS

This patent application claims priority to U.S. Patent Application Ser. No. 61/353,753 filed Jun. 11, 2010 which is incorporated by reference herein in its entirety.

### FIELD OF THE INVENTION

The present invention is directed to novel methods of identifying patients with ovarian epithelial neoplasms through the use of a metabolite probably not produced by a tumor.

### STATEMENT REGARDING FEDERALLY FUNDED RESEARCH OR DEVELOPMENT

None.

### THE NAMES OF PARTIES TO A JOINT RESEARCH AGREEMENT

None.

### REFERENCE TO SEQUENCE LISTING

None.

### BACKGROUND OF THE INVENTION

Metabolomics, one of the “(Jades tools” (e.g., genomics, transcriptomics, and proteomics) has recently emerged as an advanced technique of analytical biochemistry. Metabolomic technology is based on the detection of small molecules and the exclusion of big biopolymers such as proteins, allowing the generation of a large set of descriptors characteristic of a biological matrix. This type of methodology is used either for the “fingerprinting” of samples, allowing comparative analyses between different sample groups, or for the “(profiling” of samples in which individual differential metabolites (biomarkers) are identified for use in further targeted analysis. Clarke, C. J., et al., Metabolic Profiling as a Tool Understanding Mechanisms of Toxicity, Toxicol Pathol 36:140-7 (2008); Brindle, J. T., et al., Rapid and Noninvasive Diagnosis of the Presence and Severity of Coronary Heart Disease Using 1H-NMR-Based Metabonomics, Nat Med 8:1439-44 (2002); Coen, M., et al., An Integrated Metabonomic Investigation of Acetaminophen Toxicity in the Mouse Using NMR Spectroscopy, Chem Res Toxicol 16:295-303 (2003); Lindon, J. C., et al., Metabonomics Technologies and Their Applications in Physiological Monitoring, Drug Safety Assessment and Disease Diagnosis, Biomarkers 9:1-31 (2004); Merz, A. L., et al., Use of Nuclear Magnetic Resonance-Based Metabolomics in Detecting Drug Resistance in Cancer, Biomarkers in Medicine 3:289-306 (2009); Vallejo, M., et al., Plasma Fingerprinting with GC-MS in Acute Coronary Syndrome, Anal Bioanal Chem 394:1517-24 (2009); Lutz, U., et al., Metabolic Profiling of Glucuronides in Human Urine by LC-MS/MS and Partial Least-Squares Discriminant Analysis for Classification and Prediction of Gender, Anal Chem 78:4564-71 (2006).

While metabolomic approaches have been shown to effectively detect biochemical processes, a need exists to find metabolites which are not produced by tumors and can induce the development of a tumor as well as possibly be a target for new treatments.

### SUMMARY OF INVENTION

Provided herein are methods for identifying patients with ovarian neoplasms. The methods comprising performing an assay step of detecting a metabolite in a serum sample of a patient, wherein the metabolite is probably not produced by a tumor or could induce the development of a tumor. The presence of this metabolite is then indicative of an increased likelihood that the patient has ovarian cancer. The methods of identifying patients with ovarian epithelial neoplasms are based on high-resolution mass spectrometry. As such, the metabolites described herein are not produced by a tumor; yet it could induce the development of the tumor and further be a target for new treatments. On such metabolite useful for identifying patients with ovarian neoplasms has a molecular weight of 472 and a theoretical formula based on HCON amino acid composition of C43H65N11O13.

### BRIEF DESCRIPTION OF THE DRAWINGS

FIG. 1 shows fingerprinting by mass spectrometry using a supervised multivariate analysis as described herein.

FIG. 2 is the S-plot representation of ions detected from OPLS-DA shows the contribution of each variable to test discrimination power.

FIG. 3 shows the data of separation ovarian tumors from control patients based on Ion 472, Cut Point 6.3.

FIG. 4 provides a graphic depiction of the validation set where the cases were analyzed under identical conditions. The control cases are on the left and the ovarian tumor cases are on the right.

### DETAIL DESCRIPTION OF INVENTION

Ovarian epithelial neoplasms are usually discovered at an advanced stage. At present, measurement of serum CA-125 level and vaginal ultrasonography are the best available methods of detecting ovarian epithelial tumors. However, neither method is completely reliable for detecting ovarian epithelial cancers. For example, CA-125 serum level, which is elevated by ovarian epithelial cancer, can also be elevated by peritoneal irritation unrelated to cancer. Vaginal ultrasonography is more effective in detecting larger tumors and can give false positive results. Benjapibal, M., et al., Pre-Operative Prediction of Serum Ca125 Level in Women with Ovarian Masses, J. Med. Assoc Thai 90:1986-91 (2007); Bosse, K., et al, Screening For Ovarian Cancer by Transvaginal Ultrasound And Serum CA125 Measurement in Women with a Familial Predisposition: A Prospective Cohort Study, Gynecol Oncol 103:1077-82 (2006); Brown, P. O., et al., The Preclinical Natural History of Serous Ovarian Cancer: Defining the Target for Early Detection. PLoS Med 6:e1000114 (2009); Kalluri, M., et al., Sarcoidosis Associated with an Elevated Serum CA 125 Level: Description of a Case and a Review of the Literature, Am J Med. Sci 334:441-3 (2007); Moore, R. G., et al., How Do You Distinguish a Malignant Pelvic Mass from a Benign Pelvic Mass? Imaging. Biomarkers, or None of the Above, J Clin Oncol 25:4159-61 (2007); Podgajski, M., et al., Ascites, High CA-125 and Chronic Pelvic Pain in an Unusual Clinical Manifestation of Enterobius Vermicularis Ovarian and Sigmoid Colon Granuloma, Eur J Gynaecol Oncol 28:513-5 (2007); Romagnolo, C. et al., Preoperative Diagnosis of 221 Consecutive Ovarian Masses: Scoring System and Expert Evaluation, Eur J Gynaecol Oncol 27:487-9 (2006); Van Calster, B. et al., Discrimination Between Benign and Malignant Adnexal Masses by Specialist Ultrasound Examination Versus Serum CA-125, J Natl Cancer Inst 99:1706-14 (2007). Other researchers have proposed the use of new glycoproteins and proteomic patterns with better sensitivity and specificity for detecting ovarian carcinomas. Jackson, D., et al., Proteomic Profiling Identifies Afamin as a Potential Biomarker for Ovarian Cancer, Clin Cancer Res 13:7370-9 (12007); Visintin, I., et al., Diagnostic Markers for Early Detection of Ovarian Cancer. Clin Cancer Res, 14:1065-72 (2008); Moore, R. G., et al., The Use of Multiple Novel Tumor Biomarkers for the Detection of Ovarian Carcinoma in Patients with a Pelvic Mass, Gynecol Oncol 108:402-8 (2008).

Ovarian epithelial tumors are usually discovered at an advanced stage. CA-125 measurement and vaginal ultrasonography are typical detection methods. CA-125 is a cancer antigen or biomarker that can be quantified by analytical methods. An elevated CA-125 level indicates a possible cancer state. In addition to the cancer state, CA-125 can be elevated by peritoneal irritation, endometriosis, non-cancerous ovarian disease, and pregnancy. The quality of results from vaginal ultrasonography is dependent on lesion size. Additionally, vaginal ultrasonography may indicate a cancer state when corpus luteum, a benign, non-neoplastic lesion, is present. Highly sensitive and specific detection of the cancer state comprising ovarian neoplasms has been proposed based on detection and analysis of glycoproteins and several studies demonstrating that steroids and peptide hormones play an important role in the development of epithelial ovarian tumors have been performed. Indeed, previously, we designed two studies using only hormones and we were able to induce ovarian lesions in guinea pigs. Silva, E. G., et al., Induction of Epithelial Neoplasms in the Ovaries of Guinea Pigs by Estrogenic Stimulation, Gynecol Oncol 71:240-6 (1998); Silva, E. G., et al., The Induction of Benign Epithelial Neoplasms of the Ovaries of Guinea Pigs by Testosterone Stimulation: A Potential Animal Mode, Mod Pathol 10:879-83 (1997).

On the other hand, we have now uncovered metabolites which are unlikely to be produced by a tumor and which would identify patients with ovarian epithelial neoplasms. These metabolites could induce the development of tumors and eventually be the target for new treatments. Hence, as described herein, our focus was to uncover small metabolites in serum of patients with ovarian tumors using high-performance liquid chromatography (HPLC)-HRMS.

Specifically, we found that with 100 μL of serum, it was possible to detect between 96% and 100% of ovarian neoplasms in sample sets. Hence, the described methodology (also referred to herein as a “test”) is an excellent screening method for identifying patients with ovarian neoplasms. This test does not identify tumor markers because it identifies patients with ovarian neoplasms after the resection of the tumor—whether it is after few weeks of the resection or after several months. Our methodology can also be positive in patients who had an ovarian neoplasm, but are without evidence of disease. This means that the test identifies metabolites that are not produced by ovarian neoplasms but that are associated with ovarian neoplasms.

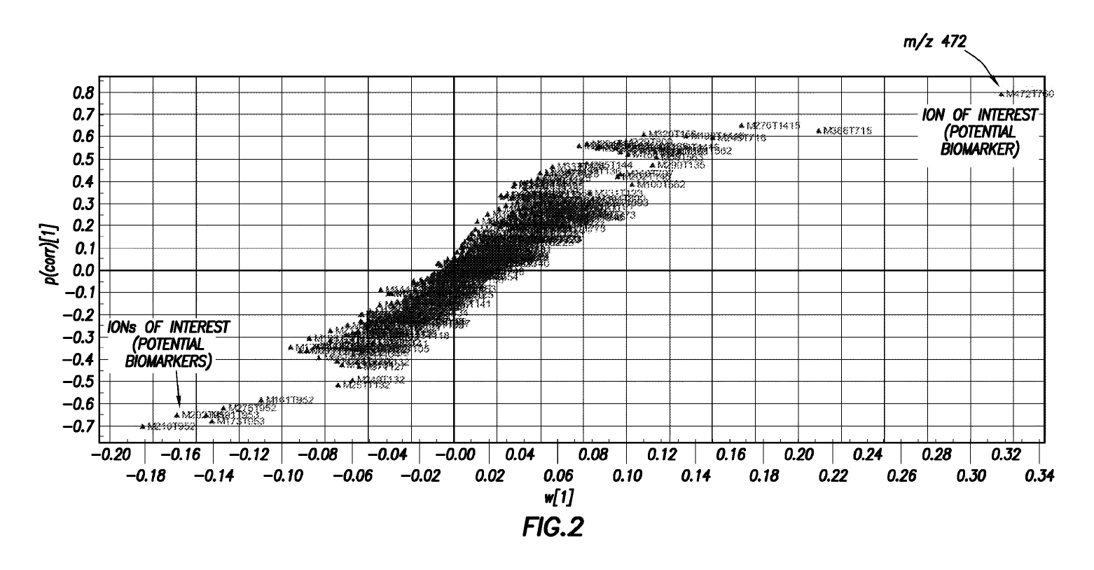

As provided by FIG. 2, the S-plot from SIMCA-P shows that ovarian neoplasms are separated from control patients based on several metabolites; however, one of the main metabolites is m/z 472. This metabolite carries the most weight at the discrimination power within the multivariate analysis. Ion 472 can induce the tumors, which, according to the genetic changes, the tumor could be benign or malignant. Therefore, Ion 472 (also referred to as “ion 472” or “m/z 427”) can be a target for therapy. The mass spectrometry information about Ion 472 shows that it is doubly charged, and therefore is a peptide. Ion 472 has a single charged molecular ion, 942.46680 and its theoretical formula based on HCON amino acid composition is C43H65N11013. The third isotope of the m/z 472 ion may indicate that the molecule contains sulfur and therefore methionine or cysteine in a peptide sequence. The MS/MS information shows that the peptide sequence contains two LL or II amino acids at the C terminus. Accurate mass immonium ion fragments confirm the presence of histidine in the peptide sequence. A database of the peptide spectra confirms the sequence HWESASLL as part of a 187 KDa protein. While this is only preliminary information, complete proteomic characterization by digestion and peptide mapping would finalize its identification. The identification of ions like Ion 472 is important in recognizing patients with ovarian neoplasms. Antibodies against these ions could be developed to block their effect on genes, which probably is the first step in the development of ovarian neoplasms.

The associated methodology described herein is based on serum fingerprints by mass spectrometry identifies women with ovarian neoplasms and provides useful information in separating women with cystadenomas and borderline tumors from women with carcinomas. Most patients with ovarian carcinomas appear to have ion 472 values of more than 7. Most patients with cystadenomas and borderline tumors have ion 472 values of less than 7. Other ions can help distinguish between women with carcinomas and women with cystadenomas and borderline tumors.

When the test described herein is combined with the CA-125 test and possibly imaging, it is possible to draw the following conclusions:
1. If our test and the CA-125 test are both positive, a tumor is present and is most likely a carcinoma. In our study, in 44 cases, metabolites by mass spectrometry recognized a neoplasm and CA-125 was elevated over 35 U/mL, 43 of these cases were carcinomas and 1 was a cystadenoma.
2. If our test and the CA-125 test are both negative, there is no ovarian neoplasm. All 59 cases where both tests gave negative results, metabolites in mass spectrometry as non-neoplastic and CA-125 below 35 U/mL were controls.
3. If there is a discrepancy between our test and the CA-125 test, imaging studies are necessary. In 35 cases, metabolites by mass spectrometry recognized an ovarian neoplasm, but CA-125 was lower than 35 U/mL. Imaging was performed in 24 of these 35 cases. Based on the results of the imaging, a diagnosis of carcinoma was rendered in 14 cases, 8 were confirmed as carcinomas, 3 were borderline and 3 were cystadenomas. Ten cases were diagnosed as benign neoplasms, all of them were cystadenomas.

There was only one case that by mass spectrometry was near the center still close to the neoplasms. For this patient, the ion intensity was 6.29, and the CA-125 level was 14 U/mL. Since the previous CA-125 taken 6 months earlier was 9, the patient underwent a vaginal ultrasound, and a small serous borderline tumor was found in an ovary. In these 79 cases, 44 in which both tests were positive and 35 discrepancies, there were no resections of the ovary for non-neoplastic conditions, such as corpus luteum.

When the results of both tests were concordant, the positive predictive value (our test positive and CA-125 >35 U/mL) was 98%, and the negative predictive value (our test negative and CA-125 <35 U/mL) was 100%. In the event of discrepancy (test positive and CA-125 <35 U/mL), imaging would be recommended. We have not seen cases with CA-125 >35 U/mL and our test negative.

In summary, this test based on serum fingerprints by mass spectrometry can identify patients with ovarian neoplasms. This test can and should be used as a screening tool for ovarian neoplasms. Patients identified as having ovarian neoplasms by our test could be further classified by CA-125 and imaging. The small molecules identified in the test proposed here do not appear to be products from the tumor.

### Example I

With the permissions of patients with ovarian tumors, we searched for small metabolites in serum of patients with ovarian tumors using high-performance liquid chromatography (HPLC)-HRMS. Table 1 provides clinical information of all cases

### Sampling Population

For the 51 patients with ovarian neoplasms, blood was collected before resection of the ovarian neoplasm in 13 patients and after resection of the ovarian neoplasm in 38 patients. Of those 38 patients, 7 patients had no evidence of residual disease and 31 patients had residual or progressive disease after resection. The 35 control patients have been obtained from a group of patients who are followed with annual physical examinations and CA-125 to determine their risk for developing ovarian neoplasms. These patients had no neoplasms of the uterus, fallopian tube or ovaries; however, 8 patients had history of breast cancer, one metastatic melanoma and one thyroid carcinoma.

Validation set: Of the 34 patients, blood samples were obtained before resection of the ovarian neoplasm for 22 patients and after resection for 12 patients; 6 of these patients had no evidence of disease at the time of blood collection. The 25 control patients in the validation set were selected from the same group as in the discovery set. In this group, 8 patients had breast cancer, one Hodgkin's lymphoma, one cervical carcinoma, one lung cancer and one had a non-neoplastic cyst of the ovary.

We searched the serum of all patients (85 with ovarian epithelial tumors and 60 healthy controls) for metabolites, including steroids and small peptides. In both the discovery and the validation cases, we compared the results of our test with the most commonly available blood test, CA-125 and with imaging.

### Reagents and Chemicals

Experimental materials included methyltestosterone (4-androstene-17αmethyl-17β-ol-3-one) and stanozolol (5α-androstan-17α-methyl-17β-ol-3,2c-pyrazole) obtained from Steraloids Ltd. (Croydon, UK). Acetic acid, ethanol, and analytical grade acetonitrile were supplied by Solvent Documentation Synthesis (SDS, Peypin, France). Water was obtained from an ultrapure water system, Nanopure, manufactured by Barnstead/Thermolyne (Thermo Scientific, Germany).

### Sample Preparation

To avoid matrix effects and preserve potentially useful small metabolites, sample preparation was designed to eliminate macromolecules. Serum samples were homogenized; subsequently, 100 μL of serum were filtered on centrifugal devices (cut off at 10 KDa) to remove high-molecular-weight proteins (9000 rpm, 4° C., 30 minutes). Filtrates (60 μL) were mixed with 20 μL of internal standard (methyltestosterone and stanozolol) in ethanol at a concentration of 1 ng/μL. After well-shaking, 10 μL of filtered serum sample were injected into the chromatographic system.

### LC-HRMS Fingerprinting

Separation of serum samples was performed on an Agilent 1200 Series HPLC system consisting of a refrigerated autosampler (set at 10° C.), a degasser, and a quaternary pump (Agilent Technologies, Waldbronn, Germany) coupled with a Finnigan LTQ-Orbitrap hybrid mass spectrometer (Thermo Fisher Scientific, Bremen, Germany). Nitrogen was produced by a Mistral-4 nitrogen generator (Schmidlin-DBS AG, Neuheim, Switzerland). An Uptisphere HDO C₁₈ column (150 mm×2.1 mm; particle size 3 μm; Interchim, Montluçon, France) fitted with a C₁₅ precolumn was used for chromatographic separation

### Data Pre-Processing

Conversion of data to a vector of peak responses (deconvolution) was done using the open-source XCMS software, freely available to use with LC-MS data. Previously, Xcalibur software was used to convert the original instrument-specific data format (*.raw) to a more common and exchangeable format (*.cdf). A report was generated showing the most statistically significant (according to P value) differences in analyte intensities as well as the respective extracted ion chromatogram for each of the first 500 most important peaks. The final results data table was imported to Microsoft Excel. The Excel file could be processed with SIMCA-P+ 12.0 (Umetrics AB, Sweden) software, and multivariate analysis was subsequently carried out.

### Statistical Analysis

Multivariate regression analysis in terms of orthogonal partial least-squares (OPLS) discriminant analysis (DA) was applied to extract the systematic variation in the quantified serum profiles (X) related to a response (Y). Trygg, J., et al., Orthogonal Projections to Latenet Structures (O-PLS), Journal of Chemometrics 16:119-28 (2002). OPLS-DA is a supervised method that uses a multiple linear regression technique to find the maximum covariance between a data set (X) and the sample class. The response y is a dummy vector describing the sample class—in our study, controls or ovarian neoplasm patients. The y vector was 1=control, and 2=ovarian neoplasm. Thus, the OPLS-DA model was performed to highlight the overall metabolic pattern related to the response (y). The robustness of the OPLS-DA model was checked by setting up a predictive model, in which ⅔ of the samples (known y) were used to predict the rest.

Variables showing a stronger correlation to y were highlighted and further investigated in the resolved data to determine whether serum metabolite concentrations differed significantly between controls and cancer patients (95% confidence level).

Results

### Discovery Set

As noted above, Table 1 provides clinical information of all cases.

TABLE 1
Clinical Information of all Cases

| | Discovery Set | Validation Set |
|---|---|---|
| Number of Patients | 86 | 59 |
| Total - 145 | | |
| Patients with Ovarian Neoplasms | 51 | 34 |
| Age | 19-77 | 38-68 |
| | Median - 59.5 | Median - 60 |
| Control Patients | 35 | 25 |
| Age | 51-78 | 51-73 |
| | Median - 61.5 | Median - 62 |
| Stage | | |
| I | 1 | — |
| II | 6 | 3 |
| III | 28 | 22 |
| IV | 4 | — |
| Unstage | 3 | — |
| Type of tumor | | |
| Ovarian carcinoma | 42 | 25 |
| Borderline tumor | 4 | — |
| Cystadenoma | 5 | 9 |

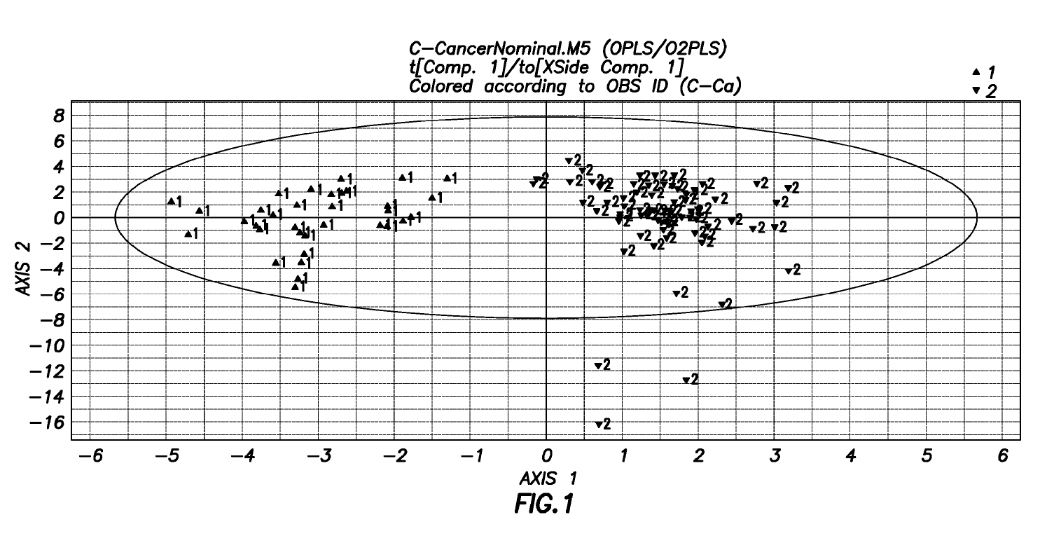

FIG. 1 provides the results of this analysis in a Score Scatter Plot. Serum finger printing by mass spectrometry using a supervised multivariate analyses is shown. The mass spectra were processed using XCMS software for background suppression, peak matching, and peak alignment. The mass spectrometry abundance obtained for each variable (ions detected) were then analyzed by Orthogonal Partial Least Square (OPLS) by means of SMICA-P software. Red (left, 1)—control cases. Green (right, 2)—ovarian tumor cases. There is a clear demarcation between both groups. Two tumors were just to the left of the 0 line. One carcinoma resected 3 month before and the patient had no evidence of disease and a 3.5 borderline tumor. The three cases under the ellipse are one carcinoma and two borderline tumors.

As shown in FIG. 1, the score scatter plot shows that 49 of the 51 ovarian neoplasms grouped in the right half of the plot (test sensitivity 96% and specificity 100%). The OPLS-DA plot of FIG. 1 shows all ovarian neoplasms grouped together, including cystadenomas, borderline, and malignant tumors. There was no difference in their location in the plot between patients from whom blood was collected before or after tumor resection, even if the patient had no evidence of disease.

### Potential Biomarkers

Our selection of potential biomarkers was based on the OPLS-DA analysis. FIG. 2 provides the S-plot representation of ions detected from our OPLS-DA analysis and the contribution of each variable to test discrimination power. The S-plot from SIMCA-P (FIG. 2) reveals the contribution of each variable to the predictive component and makes it possible to highlight variables that are the most correlated to the axis and thus represent the potentially most relevant biomarkers.

Our search for small metabolites that distinguished patients with ovarian neoplasms from controls demonstrated that the ion 471.73720 (or on 472) was present at higher levels in sera from all patients with ovarian neoplasms than in sera from most controls.

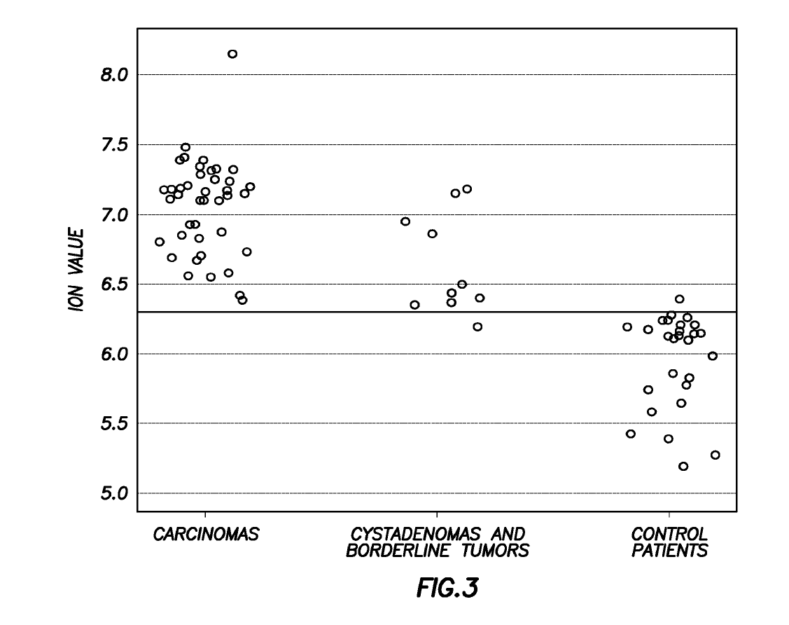

FIG. 3 shows where the value of this ion was over 6.35 in all patients with carcinomas and in all but one patient with a borderline ovarian neoplasm, while all but one control patient had an intensity below 6.35. In fact, 36 of 38 ovarian carcinoma patients (95%) had a value over 6.5. Among the cystadenomas and borderline tumors, only one patient had a value below 6.35, but 5 patients out of 10 patients had values below 6.5.

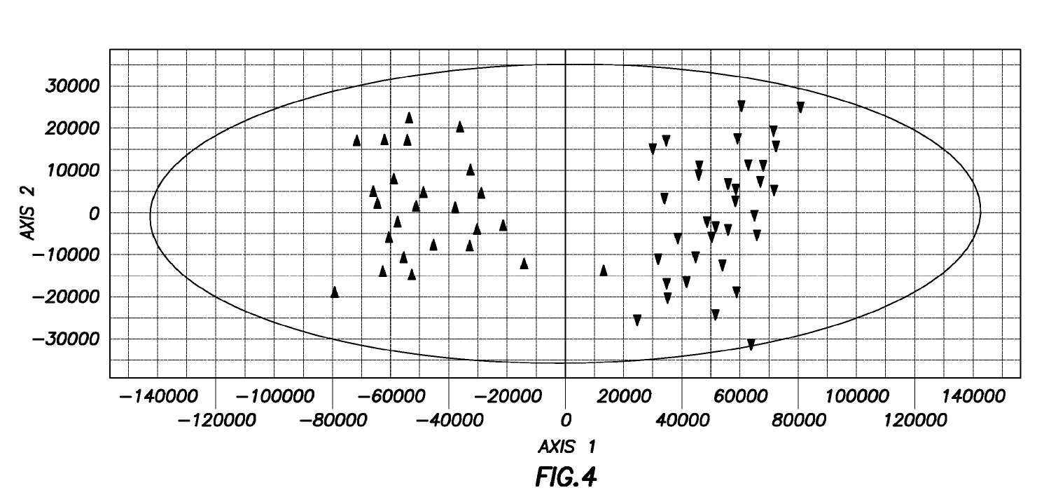

In addition, in 8 controls, the value of ion M472 was 0 (not shown in FIG. 4). The sensitivity of the test using ion M472 to identify patients with ovarian neoplasms was 98%, and the specificity was 97%. In the discovery set, CA-125 was elevated over 35 in 24 patients. FIG. 4 provides the validation set. These cases were analyzed under identical conditions as the cases included in the discovery set and described in FIG. 1. Red (left)—control cases. Green (right)—ovarian tumor cases.

### Validation Set

FIG. 4 provides the validation set. These cases were analyzed under identical conditions as the cases included in the discovery set and described in FIG. 1. Red (left)—control cases. Green (right)—ovarian tumor cases. For the 59 samples included in the validation set, preparation of the serum and use of the mass spectrometer were the same as for the discovery set. The validation set was studied using a blind approach. All 34 patients having epithelial ovarian tumors were recognized by mass spectrometry analyses, as shown in the OPLS score scatter plot obtained from HPLC-HRMS fingerprinting (FIG. 4). Twenty-four of the 25 controls were identified by mass spectrometry (test sensitivity 100% and specificity 96%). In 20 samples randomly selected from the validation set (10 ovarian neoplasms and 10 controls), we investigated the value of ion M472T760. In all 10 ovarian neoplasm samples, the intensity of M472T760 was more than 6.35, and in 9 of the 10 controls, the intensity was less than 6.35. In the validation set, CA-125 level was higher than 35 in 20 of 25 neoplasm serum samples and imaging was correct, classifying carcinomas and cystadenomas in 24 of 27 neoplasms.

We claim:

1. A method for identifying patients with ovarian neoplasms, the method comprising performing an assay step of detecting a metabolite in a serum sample of a patient, wherein the metabolite is not produced by a tumor and could induce the development of a tumor, the presence of the metabolite being indicative of the patient having an ovarian neoplasm and an increased likelihood that the patient has ovarian cancer.

2. A screening method for identifying patients with an increased likelihood of having epithelial ovarian cancer, the method comprising the steps of:
a) performing a first assay step comprising detecting in a patient's blood sample the presence of a metabolite that is not produced by a tumor and determining whether the metabolite could induce the development of a tumor; and
b) performing a second assay step if the metabolite is determined to be present in step (a) and could induce the development of a tumor, the second assay step comprising determining an elevation of the CA-125 serum level;
wherein if the presence of the metabolite and the elevation of CA-125 serum level is indicative of an increased likelihood that the patient having ovarian epithelial cancer.

3. The method of claim 1, wherein patients identified as having an increased likelihood of having ovarian epithelial cancer are subjected to additional diagnostic testing to determine if the patient has ovarian cancer, wherein the additional diagnostic testing is selected from the group consisting of pelvic examination, transvaginal ultrasound, CT scan, MRI, laparotomy, laparoscopy, and tissue sample biopsy.

4. A metabolite useful for identifying patients with ovarian neoplasms, wherein the metabolite has a molecular weight of 472 and has a theoretical formula based on HCON amino acid composition of C43H65N11O13.

5. A method of identifying a metabolite useful to identify patients with ovarian neoplasms comprising the steps collecting a statistically significant number of serum samples from patients before resection of ovarian neoplasm and after resection of ovarian neoplasm, removing macromolecules from each serum sample, separating each serum sample by HPLC and MS, and by using a multivariate regression technique, determining which metabolite concentrations differ significantly between controls and cancer patients.
